(19) United States
(12) Patent Application Publication
(10) Pub. No.: US 2008/0096257 A1
(43) Pub. Date: Apr. 24, 2008

(54) Methods for Rapid, Single-Step Strand Displacement Amplification of Nucleic Acids

(76) Inventors: Zuxu Yao, San Diego, CA (US); Graham P. Lidgard, La Jolla, CA (US)

Correspondence Address: O''Melveny & Myers LLP;IP&T Calendar Department LA-1118, 400 South Hope Street, Los Angeles, CA, 90071-2899, US

(21) Appl. No.: 11/838,024

(22) Filed: Aug. 13, 2007

Related U.S. Application Data
(60) Provisional application No. 60/837,712, filed on Aug. 15, 2006.

(51) Int. Cl. C12P 19/34

(52) U.S. Cl. 435/91.2

(57) ABSTRACT

A single-step, isothermal strand displacement amplification method that is conducted without the requirement for heat denaturation of the target nucleic acid. The method is particularly useful for analysis of clinical samples due to the decreased risk of potential contamination of the patient sample.

### RELATED APPLICATIONS

This patent application claims the priority benefit of U.S. Provisional Application Ser. No. 60/837,712, filed Aug. 15, 2006, the specification of which is incorporated herein by reference in its entirety.

### FIELD OF THE INVENTION

The invention relates to the field of molecular biology; more particularly, the invention relates to methods for single step, isothermal strand displacement amplification of target DNA. Even more particularly, the invention relates to methods for one-step strand displacement amplification of target DNA using a combination of a nicking agent and an exonuclease-deficient DNA polymerase.

### BRIEF DESCRIPTION OF THE RELATED ART

Nucleic acid amplification methods are fundamental to a wide range of scientific activities from laboratory research to clinical diagnostics. A variety of in vitro nucleic acid amplification techniques have been developed and can be loosely categorized into those requiring temperature cycling, such as polymerase chain reaction (“PCR”) and those requiring no temperature cycling, such as strand displacement amplification (“SDA”). [1,2]. SDA is based on the ability of a restriction enzyme to nick one strand of double-stranded (ds) DNA and the ability of a 5′ to 3′ exonuclease-deficient (exo⁻) DNA polymerase to extend the 3′ end from the nick. New strands extending from the 3′ ends will displace the downstream strands, which dispatch from the dsDNA as amplification products. Exponential amplification is achieved by coupling both sense and antisense reactions in which strands displaced and dispatched from a sense reaction serve as new templates for an antisense reaction and vice versa [2].

Earlier studies suggested several advantages of SDA over PCR. First, SDA has a high amplification efficiency, reaching 10¹⁰-fold of amplification in as short as 15 min [3] while PCR typically requires as long as two hours to reach an equivalent amplification level. Second, SDA is a more reliable technique for generating high molecular weight (>12 kb) genomic DNA (“gDNA”). [4]. Third, SDA is more compatible with other techniques, such as real-time diagnostic analysis of infectious and genetics diseases. [1]. Finally SDA is an isothermal amplification that can be carried out on a heat block rather than requiring a thermalcycler for accurate temperature control.

Several drawbacks, however, have hindered the general applicability of traditional SDA. First, SDA requires a heat denaturation step prior to isothermal amplification. Not all SDA enzymes, however, are heat stable (like taq polymerase in PCR). Thus, SDA enzymes must be added stepwise to the reaction after target DNA heat denaturation, thereby converting what could be an automated, single-step workflow to a manual, two-step workflow: an initial preparation step prior to heat denaturation and subsequent step necessary for addition of enzymes after heat denaturation. Importantly, the second step requires opening the reaction vessel and exposing the sample to potential contamination. This stepwise procedure is unfavorable for high throughput applications, particularly in clinical diagnostic applications in which additional exposure of the sample to the environment increases the chance of contamination.

Second, traditional SDA applications employ restriction enzymes that typically cut both strands of a target nucleic acid rather than making a nick in only one strand. Indeed, previous studies used standard restriction enzymes such as HincII [2,7,8], BSOB1 [9, 10] and Aval [11] for SDA. However, because these restriction enzymes typically cut both strands of non-denatured and unmodified gDNA, they are not good candidates for use in the one-step, isothermal SDA methods described herein. To create a comparable nick in a single strand, which is critical for SDA, non-standard nucleotides, such as α-thio-dNTP (dNTP[αS]), must be added to the reaction mixture in order to alter the enzymes' action. [2]. This will not only increase amplification cost, but also unnecessarily complicate the reaction mixture because the additional enzymes are much less efficient in nicking modified substrates, thereby leading to a slower amplification rate and lower product yield, while requiring a much higher concentration of requisite enzyme.

### SUMMARY OF THE INVENTION

The invention describes a single-step method for isothermal SDA that employs a nicking enzyme and a DNA polymerase. In preferred embodiments, the nicking enzyme is N.BbvClB and the DNA polymerase is Bst DNA polymerase. By regulating the interaction of these two enzymes, target SDA may be generated from non-denatured genomic DNA (“gDNA”) at amplification temperatures, i.e., without requiring a heat denaturation process. Thus, all reaction components, including the two enzymes, can be added simultaneously in a single step to a single reaction mixture, thereby facilitating high throughput applications. Furthermore, because reaction tubes are not opened mid-reaction, the possibility of contamination is minimized, thereby allowing SDA in clinical applications. Moreover, amplification costs are reduced due to smaller concentrations of requisite enzymes, as well as savings associated with elimination of costly dNTP[αS]. Greatly improved amplification efficiency and yields are attributable to the use of N.BbvClB.

In one embodiment, the method for isothermal strand displacement amplification of nucleic acids comprises a single step; particularly, combining, in a single reaction vessel, a mixture of: (i) double-stranded target nucleic acid; (ii) a nicking enzyme capable of nicking the double-stranded target nucleic acid; and (iii) a DNA polymerase lacking 5′-3′ exonuclease activity, under conditions sufficient to allow amplification of the target nucleic acid.

In another embodiment, the nicking enzyme is N.BbvClB and the DNA polymerase is Bst DNA polymerase, which may preferably be combined and present in the reaction mixture in equimolar concentrations. In yet another embodiment, conditions sufficient to allow amplification of the target nucleic acid include incubation at a temperature ranging from about 45° C. to about 55° C., and may preferably be conducted at a temperature of 45° C.

In sum, these improvements will make isothermal SDA an ideal new assay for a clinical setting.

### BRIEF DESCRIPTION OF THE DRAWINGS

FIG. 1 provides a schematic representation of the single-step, isolthermal SDA invention useful in different applications. A) biotin-nest primers

[symbol]

were added to reaction to convert non-biotin products to biotin-product. The biotin-nest primer, which does not contain N.BbvClB's recognition sequence, anneals to target (or non-biotin product) downstream of the 3′ end of AP

[symbol]

and is extended at 3′ end by Bst DNA polymerase to form a biotin-strand complementary to non-biotin product. The biotin-strands are eventually displaced by new strands that are extended from 3′ end of the upstream AP and dispatch as ss biotin-product. B) SDA products can be detected directly by real time probes, which remain quenched

[symbol]

in the absence of target product but emit signal

[symbol]

when hybridize to target product. Nicking enzyme N.BbvClB is show as

[symbol]

while Bst DNA polymerase is shown as

[symbol]

.

FIG. 2 is a schematic representation of biotin-product analysis on NanoChip® electronic Microarray. Biotin-products are “anchored” to streptavidin molecules in a permeation layer on a microarray (non-biotin products are unable to bind to streptavidin and are washed off the microarray). The “anchored” products are detected by discriminators (“disc”) oligos through specific hybridization between target product and a portion of the disc oligo. The other portion of disc oligo will bind to a fluorescently labeled probe (Univ.rep probe). Anchored, fluorescently labeled products bound to the microarray can be detected on a Nanogen MBW Reader. For example, the pad with all green signal (or green:red ratio>5:1) indicates a homozygous wild type (“wt”); pads with all red signals (or green:red ratio<1:5) indicate a homozygous mutant (“mut”), while pads with half green and half red indicate a heterozygous (“wt/mut”) genotype.

FIG. 3 depicts a comparison of Factor V Leiden (“FV”) amplification yields in SDA reactions that were carried out according to (A) traditional, bi-thermal SDA procedures and (B) the improved single-step, isothermal method of the present invention using different concentrations of N.BbvClB nicking enzyme and Bst DNA polymerase. Reaction number refers to one of the enzyme combinations described in Table 2, and all reactions used the same gDNA template. NTC refers to no-template-control reaction. Products were analyzed on NanoChip® electronic microarrays as described. All values are mean of two replicates. Green signals indicate wt product and red signals are for mut product. Since green:red signal ratios are >>5:1, indicating wt genotype of gDNA in reaction, the low red signal represents non-specific binding (“noise” signal) of mut reporter oligos to wt product on the microarray.

FIG. 4 shows a time course analysis of FV amplification yields in SDA reactions incubated at 50° C. for 25, 30, 35, 40 and 45 min and analyzed on a NanoChip® microarray.

FIG. 5 shows FV SNP analysis of human gDNA amplified using the single-step, isothermal SDA method of the present invention. All reactions contained 4 U N.BbvClB nicking enzyme and 4 U Bst DNA polymerase in a 36 mM K₂HPO₄ (pH7.6) buffered solution and incubated at 50° C. for 30 min. SDA products were analyzed on a NanoChip® electronic microarray.

FIG. 6 shows real-time detection of FV wt and mut product amplification from human gDNA using the single-step, isothermal SDA method of the present invention. All real-time reactions contained 4 U N.BbvClB nicking enzyme and 4 U Bst DNA polymerase in a 50 mM K₂HPO₄ (pH7.6) buffered solution and 2 fluorescence labeled probes for FV wt and mut products, respectively. Wt probe was labeled with TET and mut probe was labeled with FAM fluorescent dyes. The reactions were incubated at 45° C. and changes in fluorescent signal (both TET and FAM) were measured every 20 seconds (pseudo cycle).

FIG. 7 shows real-time SNP analysis of human gDNA amplified using the single-step, isothermal SDA method of the present invention. Real-time allele discrimination analysis was performed with the RG-3000™ software on fluorescent signal data described in FIG. 6.

FIG. 8 is a schematic representation describing a theoretical model of enzymetic generation of ssDNA templates from human gDNA and simultaneous specific target amplification from the ssDNA in SDA. The model involves three hypothetical processes: (1) Generation of ssDNA template from gDNA, i.e., a process independent of SDA primers but relying on CCTCAGC sites that are naturally present in gDNA. The CCTCAGC sites are nicked by N.BhvClB and extended at the 3′ end by Bst DNA polymerase to allow strand displacement amplification to yield ssDNA from the gDNA at incubation temperature; (2) Initiation of specific target SDA, i.e., a process where specific target primers also co-present in the reaction bind to the ssDNA templates that are generated from gDNA by SDA to initiate amplification of specific target product; and (3) Exponential target SDA, a process where newly generated target SDA products serve as new templates for more target SDA primers (sense and antisense) leading to an exponential phase of specific target amplification. All of the hypothetical processes occur simultaneously in the reaction at incubation temperature.

### DETAILED DESCRIPTION OF THE INVENTION

### Definitions

The terms “3′” and “5′” are used herein to describe the location of a particular site within a single strand of nucleic acid. When a location in a nucleic acid is “3′ to” or “3′ of” a reference nucleotide or a reference nucleotide sequence, this means that the location is between the 3′ terminus of the reference nucleotide or the reference nucleotide sequence and the 3′ hydroxyl of that strand of the nucleic acid. Likewise, when a location in a nucleic acid is “5′ to” or “5′ of” a reference nucleotide or a reference nucleotide sequence, this means that it is between the 5′ terminus of the reference nucleotide or the reference nucleotide sequence and the 5′ phosphate of that strand of the nucleic acid. Further, when a nucleotide sequence is “directly 3′ to” or “directly 3′ of” a reference nucleotide or a reference nucleotide sequence, this means that the nucleotide sequence is immediately next to the 3′ terminus of the reference nucleotide or the reference nucleotide sequence. Similarly, when a nucleotide sequence is “directly 5′ to” or “directly 5′ of” a reference nucleotide or a reference nucleotide sequence, this means that the nucleotide sequence is immediately next to the 5′ terminus of the reference nucleotide or the reference nucleotide sequence.

A “naturally occurring nucleic acid” refers to a nucleic acid molecule that occurs in nature, such as a full-length genomic DNA molecule or an mRNA molecule.

An “isolated nucleic acid molecule” refers to a nucleic acid molecule that is not identical to any naturally occurring nucleic acid or to that of any fragment of a naturally occurring genomic nucleic acid spanning more than three separate genes.

As used herein, “nicking” refers to the cleavage of only one strand of a fully double-stranded nucleic acid molecule or a double-stranded portion of a partially double-stranded nucleic acid molecule at a specific position relative to a nucleotide sequence that is recognized by the enzyme that performs the nicking. The specific position where the nucleic acid is nicked is referred to as the “nicking site.”

A “nicking agent” is an enzyme that recognizes a particular nucleotide sequence of a completely or partially double-stranded nucleic acid molecule and cleaves only one strand of the nucleic acid molecule at a specific position relative to the recognition sequence.

A “nicking endonuclease,” as used herein, refers to an endonuclease that recognizes a nucleotide sequence of a completely or partially double-stranded nucleic acid molecule and cleaves only one strand of the nucleic acid molecule at a specific location relative to the recognition sequence. Unlike a restriction endonuclease, which requires its recognition sequence to be modified by containing at least one derivatized nucleotide to prevent cleavage of the derivatized nucleotide-containing strand of a fully or partially double-stranded nucleic acid molecule, a nicking endonuclease typically recognizes a nucleotide sequence composed of only native nucleotides and cleaves only one strand of a fully or partially double-stranded nucleic acid molecule that contains the nucleotide sequence.

An “amplification primer,” as used herein, is an oligonucleotide that anneals to a template nucleic acid comprising a sequence of an antisense strand nucleic acid and functions as a primer for an initial primer extension. The resulting extension product from the initial primer extension, that is, the strand containing the nucleotide of the amplification primer, is then nicked and the fragment in the same strand containing the 3′ terminus at the nicking site serves as a primer for subsequent primer extensions.

The present invention describes a novel method that enables SDA reactions to be conducted without disrupting workflow and without heat denaturation of target neucleic acid. Indeed, this single-step, isothermal SDA reaction method does not require any sophisticated treatment, but instead utilizes reactants already and otherwise present in a typical SDA reaction mixture. While not wishing to be bound by a particular theory, the method appears to operate based on interactions of two SDA enzymes in conjunction with the naturally present CCTCAGC sequence in human gDNA. When added to reaction, the CCTCAGC sites in non-denatured gDNA are recognized and nicked by N.BbvClB. New strands are then extended from 3′ ends of the nicks by Bst polymerase present in reaction to displace the downstream strands that form ssDNA which immediately serve as templates for SDA primers that are also present in the system leading to initiation of specific target amplification, all occurring simultaneously. FIG. 8 presents a schematic model of the process. The present invention allows all SDA reactants, including the two enzymes, to be added to reaction tubes in a single step as is routinely done for PCR. The improved workflow, makes the technique particularly useful for high throughput and clinical applications.

Although ssDNA can be produced from non-denatured gDNA in SDA reactions, production of ssDNA is correlated to the concentration of SDA enzymes present in the reaction tube. In the one-step, isothermal SDA, amplification occurred primarily in tubes containing 4 U of N.BbvClB (#4, 7 and 10) and not in tubes that had less N.BbvClB enzyme. The decreased SDA amplification in these reactions may be due to a failure in generating ssDNA from gDNA, as opposed to target amplification from the ssDNA because all those enzyme mixes with less N.BbvClB were able to produce strong SDA when ssDNA had been already generated, e.g., through heat denaturation as demonstrated in bi-thermal SDA. The best results were achieved when the N.BbvClB:Bst polymerase enzymes were in an activity ratio of about 1:1 to about 1:2.

Unlike PCR which typically produces a single species of product, SDA results in amplification products of differing lengths. This makes analysis on agarose gel to pinpoint specific SDA products difficult. [12]. Thus, choosing the best technique to accurately identify correct product from a product mix is important for SDA reactions. Two techniques have proven useful with the single-step, isothermal SDA method of the present invention.

The first technique incorporates the NanoChip® electronic microarray, which has been previously described. [5,6,13,14]. Because analysis on NanoChip® electronic microarrays requires biotin-product, while SDA typically produces non-biotin product, biotin-nest primers have been used with the one-step, isothermal SDA reaction to “catch” and “convert” the non-biotin SDA products to biotin-products. Successful demonstration of this conversion is demonstrated herein and has been described previously. [11].

The second technique incorporates real-time product analysis. Real-time SDA was developed to accommodate two ubiquitous fluorescence-labeled FV probes that were primarily designed for a real-time PCR. Both probes demonstrated low backgrounds in the absence of target but high specificity and affinity to their targets due to the incorporation of an Eclipse™ Dark Quencher, the MGB™ technology, and modified bases, such as Super A and Super T. With the single-step, isothermal, real-time SDA developed with these two probes, rapid analysis of SNP clinical samples was demonstrated. It is worthy of note that incubating at 45° C. is preferred for the presently claimed single-step, isothermal real-time SDA. The real-time data confirm that single-step, isothermal SDA amplification is very efficient, allowing real-time SNP analysis to occur in as few as 10 min, greatly surpassing both bi-thermal, real-time SDA described previously [9,15,16] and all real-time PCR analyses known to the inventors.

Methods and Compositions for Isothermal SDA

SNP analysis of the Factor V Leiden (“FV”) gene was used as a test model to demonstrate feasibility of the single-step, isothermal SDA method of the present invention for clinical applications. Those of skill in the art will appreciate that the presently claimed method is generally applicable for genomic target amplification. Indeed, because the recognition sequence for N.BbvClB, CCTCAGC, is present in all genes throughout the entire genome, the reaction should generate a pool of ssDNA products from every gene. Once generated, this pool of ssDNA will serve as templates for designated targets whose primers are present in reaction. Moreover, experiments using the single-step, isothermal SDA method of the present invention to amplify the prothrombin gene, either in monoplex or biplex amplification with the FV gene have been successful. The combination of 4 U N.BbvClB nicking enzyme and 4 U Bst DNA polymerase also produced the best result.

Genomic DNA (“gDNA”) was prepared from human whole blood from San Diego Blood Bank (San Diego, Calif., USA) using Qiagen Midi DNA Kit (Qiagen, Valencia, Calif., USA) and stored at −20° C. until use. Genotypes of the DNA samples used in this study were determined by SNP analysis on a Nanogen Molecular Biology Workstation.

Nicking endonuclease N.BbvClB and Bst DNA polymerase (Large Fragment) were purchased from New England Bio-labs (Beverly, Mass., USA). Sequences of oligonucleotides useful in the practice of the invention are described in Table 1.

TABLE 1
Oliginucleotides useful in this invention

| | |
|---|---|
| Oligo name | Sequence (5′-3′) |

Primers:

| | |
|---|---|
| FV forward AP | 5′-CATCATGAGAGACATCGCCTCCTCAGCAATAGGACTAC-3′ |
| FV reverse AP | 5′-AAATTCTCAGAATTTCTGAACCTCAGCTTCAAGGACAA-3′ |
| FV reverse bumper | 5′-GCCCCATTATTTAGCCAGGA-3′ |
| FV nest primer | 5′-bio-TGTAAGAGCAGATCCCTGGAC-3′ |

Real time detection probes#

FAM-AGGCAAGGA*AT*A*C-exon; mutant (Gln)
TET-AGGCGAGGA*AT*A*C-exon; wild type (Arg)

Reporters:

| | |
|---|---|
| FV Wt disc | 5′-CTGAGTCCGAACATTGAGTCCTGTATTCCTCG-3′ |
| FV Mut disc | 5′-GCAGTATATCGCTTGACATCCTGTATTCCTTG-3′ |
| FV stab | 5′CCTGTCCAGGGATCTGCTCTTAC 3′ |
| WT univ rep probe | 5′-CTCAATGTTCGGACTCAG-A532 |
| MUT univ rep probe | 5′-TGTCAAGCGATATACTGC-A647 |

#Synthesized at Nanogen North (Bothell, WA, USA);
*indicates superbase structure in oligonucleotide;

Bold and underlined letter indicates SNP base

Both forward and reverse amplification primers contain a recognition sequence CCTCAGC (underlined) for N.BbvClB. Because these two primers, when fully matched to complementary strands, are nicked by N.BbvClB at the recognition site to allow generation of multiple copies of product from a single primer (i.e., “amplifiable”), they are termed amplification primers (AP). The bumper primer does not contain the recognition sequence (thus “non-amplifiable”). Other oligonucleotides in Table 1 are useful for converting SDA product to biotinylated (biotin-) product (FV nest primer), for real-time product detection and for preparing product detection reporters. Unless specified separately, all oligonucleotides in Table 1 were synthesized in the Integrated DNA Technologies (IDT, Coralville, Iowa, USA).

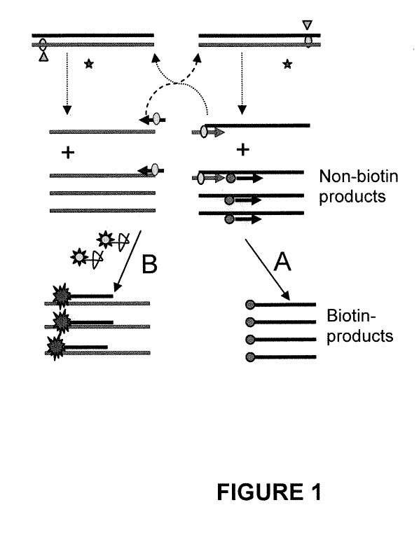

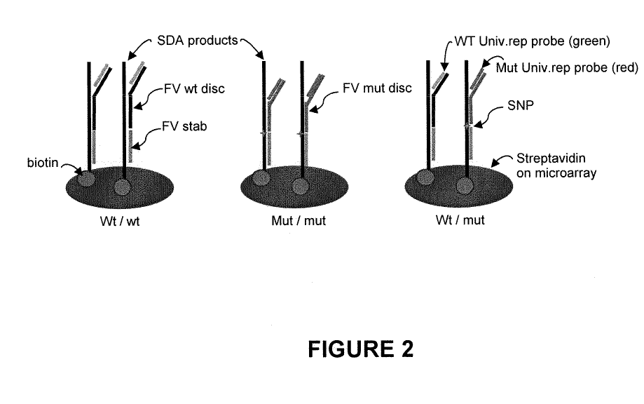

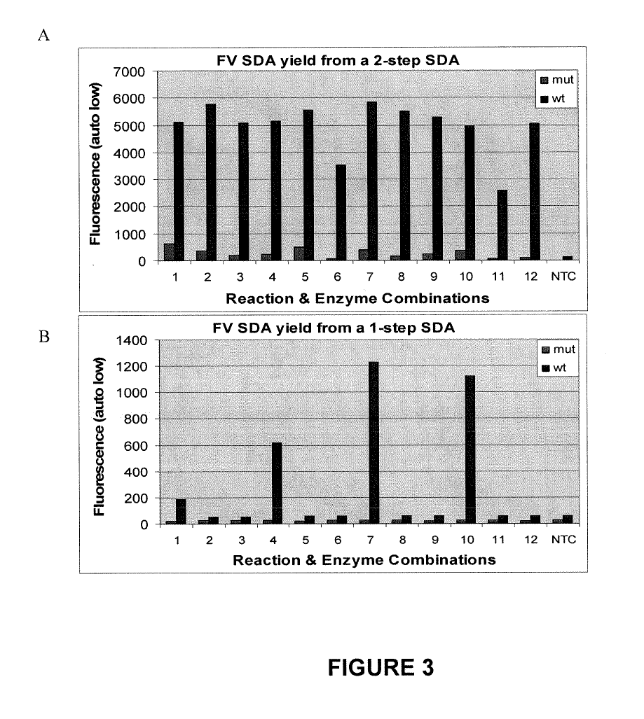

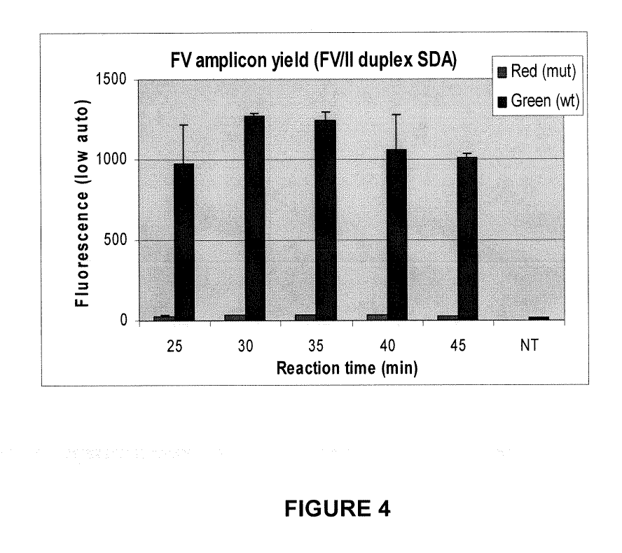

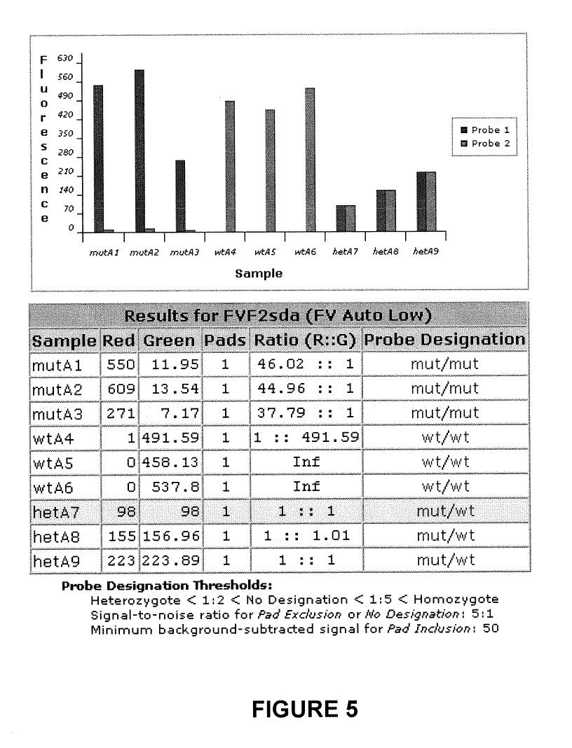

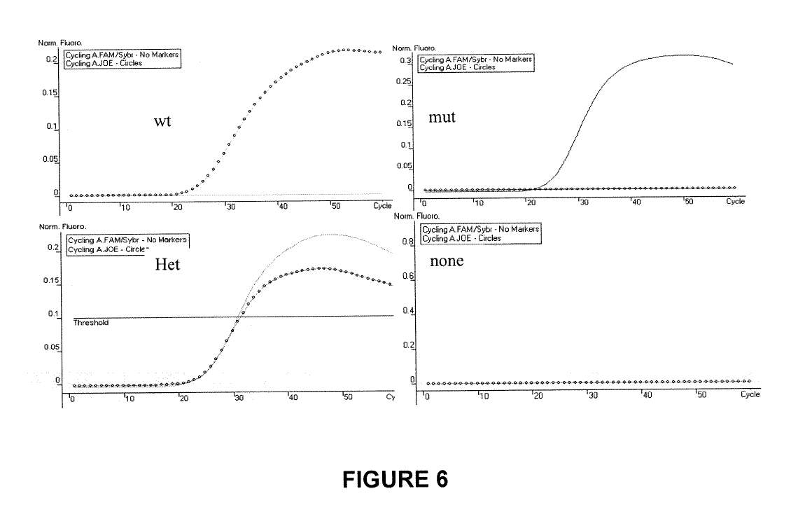

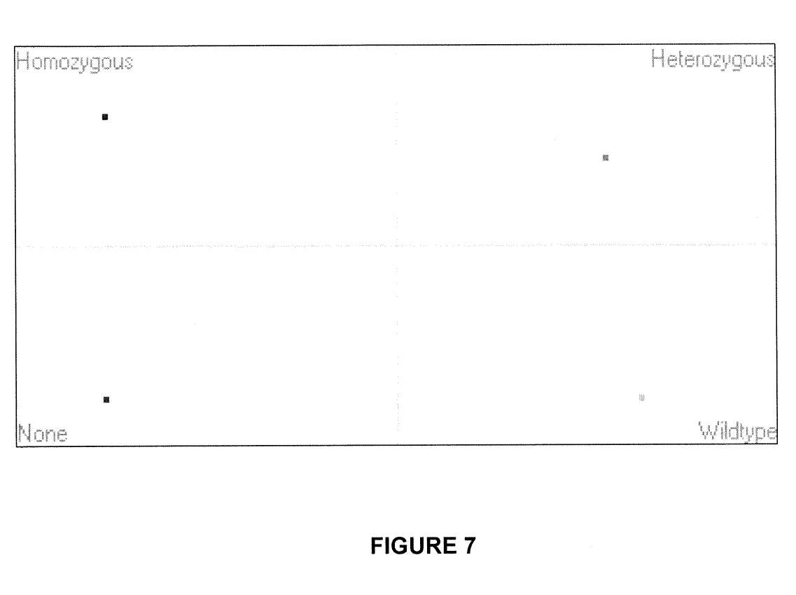

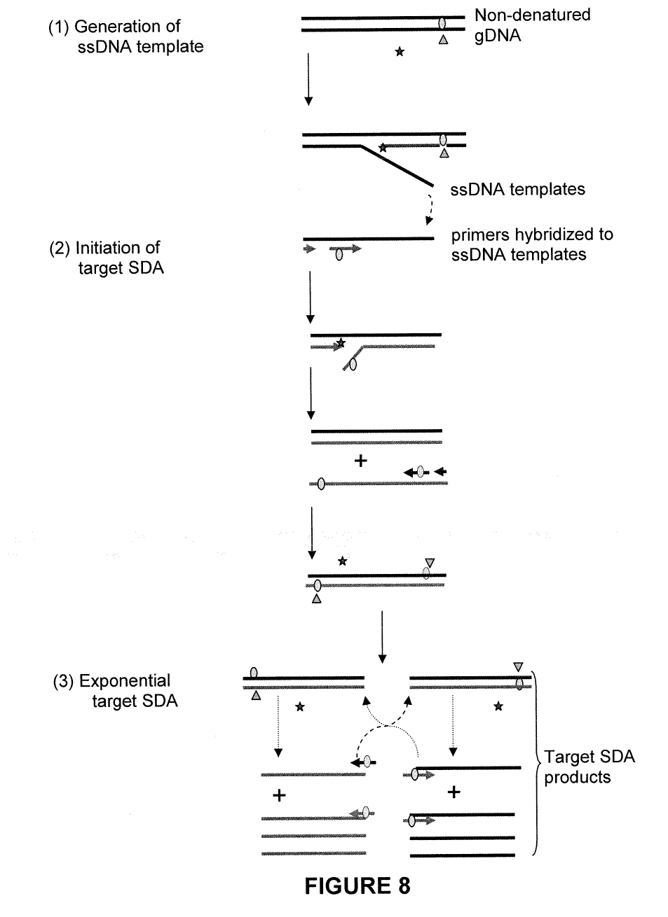

For analysis on NanoChip® electronic microarrays, reactions were performed in a 10 μL final volume containing 50 ng human gDNA, 250 nM forward and reverse AP, 25 nM reverse bumper and 500 nM nest primer, 3.75 mM MgCl₂, 36 mM K₂HPO₄ (pH7.6), 0.25 mM each dNTPs, 4 U N.BbvClB and 4 U Bst DNA polymerase diluted in Diluent A (New England Biolabs, Beverly, Mass., USA). All components, including the two enzymes, were added to a 200-μL microcentrifuge tube at room temperature. The tube subsequently proceeded directly to incubation on a 50° C. heat block for 30 min (no initial target heat denaturation step was involved). Addition of nest primers converts non-biotin product to biotin-products as shown in FIG. 1. After 30 min of incubation, the reactions were diluted 60 fold in 60 μL of 50 mM histidine and electronically addressed on the Nanogen Molecular Biology WorkStation (MBW) Loader to a NanoChip® electronic microarray where biotin-products attached to streptavidin molecules in the permeation layer, while non-biotin products were washed off the microarray. The products attached to the microarray were then detected by two fluorescence labeled probes using two discriminator oligonucleotides, as shown in FIG. 2. The level of fluorescent signal detected on the microarray represents the yield of target product (green signal for wild type and red signal for mutant products) while a green:red signal ratio determines genotype of the amplification product. Details of product detection and SNP analysis on NanoChip® electronic microarray have been described elsewhere. [5, 6].

A real-time assay was developed to confirm product amplification resulting from the improved one-step SDA of the present invention. The real-time reaction was also run in a 10 μL final volume having a similar composition to that described above (50 ng gDNA, 250 nM FV forward and reverse AP, 25 nM FV reverse bumper, 3.75 mM MgCl₂, 50 mM K₂HPO₄, pH7.6, 0.10 mM each dNTPs, 4 U N.BbvClB and 4 U Bst DNA polymerase) plus 0.5 μL of a 20 fold concentrated probe solution that contains two fluorescence labeled probes specific to wild type and mutant Factor V Leiden (“FV”) products, respectively (Table 1). No nest primer was used in this assay. Fluorescence on the probes is quenched in the absence of target products but is emitted when the probes hybridize to their targets, as shown in FIG. 1. The reactions were prepared at room temperature and incubated at 45° C. on a Rotor-Gene 3000™ Four-Channel Multiplexing System (Corbett Robotics of Australia). No initial target heat denaturation step was required. The fluorescent signals in each reaction were collected every 20 seconds during incubation and analyzed by the RG-3000™ software for allele discrimination.

Additional embodiments of the invention are described in the Examples below.

### EXAMPLES

### One-Step, Isothermal SDA Reactions

The nicking enzyme, N.BbvClB, and Bst DNA polymerase are useful to exemplify the one-step, isothermal SDA method of the present invention. Skilled artisans, however, will recognize that other combinations of nicking enzymes and exonuclease-deficient DNA polymerases can be used to practice the claimed invention. Accordingly, the invention should not be understood to be limited to nicking enzyme, N.BbvClB, and Bst DNA polymerase.

To understand the role of each enzyme and their interactions in SDA, twelve combinations were prepared of the two enzymes as shown in Table 2.

| Enzyme | N.BbvC1B, | N.BbvC1B, | N.BbvC1B, |
|---|---|---|---|
| Dilution, total units in reaction | 1x, 4U | ½x, 2U | ¼x, 1U |
| Bst DNA polymerase, 1x, 29U | #1 (4U:29U) | #2 (2U:29U) | #3 (1U:29U) |
| Bst DNA polymerase, ½x, 14.5U | #4 (4U:14.5U) | #5 (2U:14.5U) | #6 (1U:14.5U) |
| Bst DNA polymerase, ¼x, 7.25U | #7 (4U:7.25U) | #8 (2U:7.25U) | #9 (1U:7.25U) |
| Bst DNA polymerase, ⅛x, 3.6U | #10 (4U:3.6U) | #11 (2U:3.6U) | #12 (1U:3.6U) |

*all enzyme levels were determined in 2 μL of Diluent A from New England Bio-labs (Beverly, MA, USA).

A master mix containing all SDA reaction components, except the two enzymes, was prepared and aliquoted (8 μL each) to individual reaction tubes. The first set of 12 tubes was subjected to a bi-thermal amplification procedure, i.e., reaction tubes were first heated to 95° C. for 5 min and returned to 50° C. Then, after reaching 50° C., 2 μL of enzyme mix from Table 2 was added to each tube and incubated at 50° C. for 30 min. As shown in FIG. 3A, the resulting amplification products were analyzed on a NanoChip® electronic microarray, which showed similar amplification patterns from all tubes regardless of the different concentrations or combination of the two enzymes in each reaction. Wild-type FV was used as the gDNA test sample used for this test.

The second set of tubes was not subjected to heat denaturation at 95° C. To each tube, 2 μL of enzyme mix from Table 2 was added at room temperature. All tubes were incubated at 50° C. for 30 min. As shown in FIG. 3B, without the initial 95° C. treatment, most reactions did not result in any SDA product as expected. The exceptions were tubes numbered 4, 7 and 10, in which strong product amplification were detected. Repeated tests demonstrated that a combination of 4 U N.BbvClB with 4 U Bst DNA polymerase (tube #10) in a 10 μL reaction produced the best SDA result. Thus, under the appropriate certain circumstance (e.g., the proper combination of the two enzymes in reaction), SDA can be initiated without a heat denaturation step.

To determine the amplification time required for SDA, reactions were prepared and incubated at 50° C. for 25, 30, 35, 40 and 45 min, respectively. FIG. 4 shows that amplification reached significant levels in 25 min and peaked in 30 min of incubation. Sufficient quantities of amplification product were obtained to conduct analysis on the NanoChip® electronic microarray.

SNP Analysis

Because the improved, single-step, isothermal SDA resulted in strong target amplifications, the feasibility of using the method for SNP analysis was investigated. SNP analysis was tested using 9 human genomic samples, consisting of three known FV wild type (WT), three FV mutant (Mut) and three FV heterozygotes (Het) samples. The single-step reactions were prepared as described and incubated at 50° C. for 30 min (without the initial heat denaturation step). FIG. 5 shows SNP analysis results on a NanoChip® electronic microarray. All 9 samples matched correctly to their known genotypes, demonstrating that the improved SDA technique can be used for human genomic sample amplification and SNP analysis.

Real-Time One-Step, Isothermal SDA Reactions

FIG. 6 shows real-time changes of fluorescent signals from four single-step SDA reactions, each containing a FV WT, a Mut, a Het sample or no template (NT), respectively. Fluorescent signal was not observed in the first 20 cycles (or 6.7 min) but reached mid-log phase in 30 cycles (10 min) and plateau in 50 cycles (17 min) in all reactions except in NT reaction where fluorescent signal was not detected throughout the reaction. The detection of real-time signals confirms the presence and production of specific target products in reaction because only probes binding to their specific targets would result in fluorescent emission. Thus, the real time SDA confirms that the one-step, isothermal SDA technique amplified all targets correctly, because only TET signals (from probe for WT product, read in Joe channel with excitation source at 530 nm and detection filter 555 nm) were detected in reaction with WT sample, only FAM signals (from probe for MUT product, with excitation source at 470 nm and detection filter 510 nm) were detected in reaction with MUT sample, and both TET and FAM signals were detected in reaction with HET samples. Genotype analysis was achieved from the one-step, isothermal real-time SDA reaction, as shown in FIG. 7.

Although the foregoing invention has been described in some detail by way of illustration and example for purposes of clarity and understanding, it may be readily apparent to those of ordinary skill in the art in light of the teachings of this invention that certain changes and modifications may be made thereto without departing from the spirit or scope of the appended claims.

### REFERENCES

1. Schweitzer B, Kingsmore S. (2001) Current Opinion in Biotechnology, 12:21-27.
2. Walker G T, Little M C, Nadeau J G, Shank D D. (1992) Proc.Natl.Acad.Sci.USA, 89:392-396.
3. Hellyer T J, Nadeau J G. (2004) Expert Rev Mol Diagn. 4(2):251-61.
4. Luthra R, Medeiros L J. (2004) J Mol Diagn., 3:236-242.
5. Evans J G, Lee-Tataseo C. (2002) Clinical Chemistry, 48(9):1406-1411.
6. Erali M, Schmidt B, Lyon E, Wittwer C. (2003) Clinical Chemistry, 49(5):732-739.
7. Walker G T, Linn C P, Nadeau J G. (1996) Nucleic Acids Research, 24(2):348-353.
8. Badak F Z, Kiska D L, O'Connell M, NYCZ C M, Hartley C, Setterquist S, Hopfer R L. (1997) J. Clinical Microbiology, p 1239-1243.
9. Nadeau J G, Pitner J B, LIN C P, Schram J L. Dean C H, Nycz C M. (1999) Analytical Biochemistry, 276(2):177-187.
10. Westin L, Xu X, Miler C, Wang L, Edman C F, Nerenberg M. (2000) Nature Biotechnology, 18:199-204.
11. Huang Y, Shirajian J, Schroder A, Yao Z, Summers T, Hodko D, Sosnowski R. (2004) Electrophoresis, 25:000-000.
12. Walker G T. (1993) PCR Methods Appl, 3(1):1-6.
13. Tsang S, Sun Z, Stewart C, Lum N, Frankenberger C, Subleski M, Rasmussen L, Munroe D J. (2004) Biotechniques, 36(4):682-688.
14. Moutereau S, Narwa R, Matheron C, Vongmany N, Simon E, Goossens M. (2004) Hum Muta., 23(6):621-628.
15. Ugozzoli L A, Chinn D, Hamby K. (2002) Analytical Biochemistry, 307:47-53.
16. Wang S S, Thornton K, Kuhn A M, Nadeau J G, Hellyer T J. (2003) Clinical Chemistry, 49(10):1599-607.

What is claimed is:

1. A method for isothermal strand displacement amplification of nucleic acids, the method comprising the step of:
combining, in a single reaction vessel, a mixture of:
i. double-stranded target nucleic acid;
ii. a nicking enzyme capable of nicking the double-stranded target nucleic acid; and
iii. a DNA polymerase lacking 5′-3′ exonuclease activity; under conditions sufficient to allow amplification of the target nucleic acid.

2. The method of claim 1, wherein the nicking enzyme is N.BbvClB and the DNA polymerase is Bst DNA polymerase.

3. The method of claim 2, wherein the N.BbvClB and Bst DNA polymerase are present in an approximately equimolar amount.

4. The method of claim 3, wherein the N.BbvClB and Bst DNA polymerase are present at a concentration of about 4 U each.

5. The method of claim 1, wherein the conditions sufficient to allow amplification of the target nucleic acid include incubation at a temperature ranging from about 45° C. to about 55° C.

6. The method of claim 4, wherein the conditions sufficient to allow amplification of the target nucleic acid include incubation at about 45° C.

7. The method of claim 1, wherein the target nucleic acid is genomic DNA.

8. The method of claim 7, wherein the genomic DNA is obtained from a patient in need of a clinical diagnosis.

9. A method for isothermally amplifying a target nucleic acid, the method comprising the steps of:
a. providing a target double-stranded nucleic acid containing the target nucleic acid sequence and a sequence capable of being recognized by a nicking enzyme;
b. providing a nicking enzyme;
c. nicking the target double-stranded nucleic acid with the nicking enzyme to provide at least two new 3′ termini in the nucleic acid;
d. providing a DNA polymerase lacking 3′-5′ exonuclease activity;
e. extending one or more of the at least two new 3′ termini with the DNA polymerase thereby producing a newly synthesized strand; and
f. repeating the nicking and extending steps such that the target nucleic acid sequence is amplified
under conditions sufficient to allow amplification without addition of external deoxynucleoside triphosphate moieties.

10. The method of claim 9, wherein the nicking enzyme is N.BbvClB and the DNA polymerase is Bst DNA polymerase.

11. THe method of claim 9, wherein the conditions sufficient to allow amplification of the target nucleic acid include incubation at a temperature ranging from about 45° C. to about 55° C.


SEQUENCE LISTING (11 sequences)

SEQ ID NO: 1 - DNA, 38 bp, Homo sapien
catcatgagagacatcgcctcctcagcaataggactac

SEQ ID NO: 2 - DNA, 38 bp, Homo sapien
aaattctcagaatttctgaacctcagcttcaaggacaa

SEQ ID NO: 3 - DNA, 20 bp, Homo sapien
gccccattatttagccagga

SEQ ID NO: 4 - DNA, 21 bp, Homo sapien
tgtaagagcagatccctggac

SEQ ID NO: 5 - DNA, 13 bp, Homo sapien
aggcaaggaatac

SEQ ID NO: 6 - DNA, 13 bp, Homo sapien
aggcgaggaatac

SEQ ID NO: 7 - DNA, 32 bp, Homo sapien
ctgagtccgaacattgagtcctgtattcctcg

SEQ ID NO: 8 - DNA, 32 bp, Homo sapien
gcagtatatcgcttgacatcctgtattccttg

SEQ ID NO: 9 - DNA, 23 bp, Homo sapien
cctgtccagggatctgctcttac

SEQ ID NO: 10 - DNA, 19 bp, Homo sapien
ctcaatgttcggactcaga

SEQ ID NO: 11 - DNA, 18 bp, Homo sapien
tgtcaagcgatatactgc